(19) United States
(12) Patent Application Publication
(10) Pub. No.: US 2013/0065290 A1
(43) Pub. Date: Mar. 14, 2013

(54) COMBINED DRY AND WET DUAL PHASE ANAEROBIC PROCESS FOR BIOGAS PRODUCTION

(71) Applicants: Nitant Vishnu Mate, Pune (IN); Devendra Jayant Goyal, Bibvevadi (IN); Ashwin Sharad Joshi, Phaltan (IN); Nikhil Appasaheb Khot, Kolhapur (IN); Shirish Madhav Ganu, Pune (IN)

(72) Inventors: Nitant Vishnu Mate, Pune (IN); Devendra Jayant Goyal, Bibvevadi (IN); Ashwin Sharad Joshi, Phaltan (IN); Nikhil Appasaheb Khot, Kolhapur (IN); Shirish Madhav Ganu, Pune (IN)

(73) Assignee: Kirloskar Integrated Technologies Limited, Pune (IN)

(21) Appl. No.: 13/520,290

(22) PCT Filed: Jan. 4, 2011
(86) PCT No.: PCT/IN2011/000002

§ 371 Date: Nov. 28, 2012

(30) Foreign Application Priority Data
Jan. 4, 2010 (IN) 015/MUM/2010

(51) Int. Cl. C12P 5/02

(52) U.S. Cl. 435/167

(57) ABSTRACT

The present invention encompasses a self sustaining and combined dual biomethanation process to produce biogas and manure. The said biomethanation process comprises two or more different biogas reactors using mixed and/or multiple solid biomass as feed. The leachates generated from the solid digester are utilitzed by recirculating the leachates produced, thereby ensuring optimum biogas generation.

### FIELD OF INVENTION

This invention relates to a method of biogas generation.

Particularly, the present invention relates to a dual anaerobic fermentation process for biogas generation using microbial consortia.

More particularly the present invention relates to a combined dual anaerobic fermentation process for biogas generation and in two or more different anaerobic digesters.

### GLOSSARY

### Biomethanation:

Biomethanation is the formation of methane, a metabolic by product in anoxic conditions by microbes known as methanogens under anaerobic condition.

### Biomass:

Biomass is defined as the total amount of living material in a given habitat. Herein biomass is referred to as any carbonaceous organic substrate including, but not limited to, sewage sludge, forestry waste, food waste, agricultural waste, municipal waste, agricultural feeds, agricultural produce, and the like.

### Dual Process of Biomethanation:

Herein defined as a biomethanation process in solid phase/state as one step and biomethanation process in liquid phase/state as the other step

### Hydraulic Retention Time (HRT):

The hydraulic retention time (HRT) is a measure of the average length of time that a substance/material/compound remains in a constructed reactor.

### Methane Digester:

Methane digesters are anaerobic (low or no oxygen) chambers which facilitate the breakdown of manure (substrate) by anaerobic bacteria with the release of methane and other, gases as byproducts of their metabolism, including ammonia, nitrogen, hydrogen sulfide, and sulfur dioxide. Herein substrate used is any biomass as defined above.

### Liquid State Methane Digester:

Herein defined as a methane digester wherein the leachates from solid state methane digester, fresh culture and feed are in liquid state, i.e. in a flowable form.

### Solid State Anaerobic Fermentation:

A process of anaerobic fermentation wherein the contents of the digester are in a non pump-able i.e. dry form—it may have considerably high percentage of liquid absorbed in the solid mass.

### Solid State Methane Digester:

Herein defined as a methane digester wherein culture, feed is in a moist but solid state i.e. the contents of the digester are in non pump-able form.

### Total Solids (T.S.):

The total content of suspended and dissolved solids in liquid

### BACKGROUND OF THE INVENTION

Anaerobic digestion is a biological process to degrade organic matter to produce biogas which is a renewable energy source and a sludge that could be used as fertilizer. In the absence of oxygen (anaerobic digestion), the organic matter is degraded partially by the combined action of several types of micro-organisms. A succession of biological reactions takes place leading to the formation of biogas and sludge. The bacteria which carry out these reactions exist in natural state in the liquid manure and the anaerobic ecosystems; it is not necessary to add more, they grow and multiply naturally in a medium without oxygen.

Anaerobic digestion is a series of processes in which biodegradable material is broken down by microorganisms and biochemical processes in the absence of oxygen and is widely used to treat wastewater. Anaerobic digestion is also widely used as a renewable energy source because the process produces methane rich biogas suitable for use as a source of energy helping replace fossil fuels as also, the nutrient-rich digestate can be used as fertilizer. The digestion process begins with bacteria assisted hydrolysis of the biomass materials to break down insoluble organic polymers such as carbohydrates, proteins, lipids and the like into some variety of sugars and/or amino acids, and make them available for another consortium of bacteria. Acidogenic bacteria then convert the sugars and amino acids into carbon dioxide, hydrogen, ammonia, and organic acids. Acetogenic bacteria further convert these resulting organic acids primarily into acetic acid and partially into other volatile fatty acids, along with additional ammonia, hydrogen, and carbon dioxide. Methanogens, finally convert these products to methane and carbon dioxide. This conversion is brought about by various processes and using substrates in different forms, phases i.e. wet and dry. Dry phase fermentation solves the scum formation problems as like created in the wet phase process of biomethanation. The slurry developed after biomethanation process is let out or is dumped out, hereby creating lose of microbes which succeeding can be utilized for biogas generation and manure production, and creating minimum amounts of than produced slurry and/or sludge.

Utilizing anaerobic digestion technologies help to reduce the emission of greenhouse gasses in a number of ways:
Replacement of fossil fuels
Reducing methane emission from landfills
Displacing industrially-produced chemical fertilizers
Reducing electrical grid transportation losses; as the electricity produced by a biogas plant is invariably consumed by localized consumers.

Herein is an option developed to overcome the above mentioned limitations with regards to single phase digestion biomethanation process. Some of the current technologies available for anaerobic digestion and their shortcomings are as follows.

### PRIOR ART

1) Patent Application (WO/2007/096392) discloses “BIOREACTOR FOR METHANIZATION OF BIOMASS HAVING A HIGH SOLIDS FRACTION.”

Abstract: A bioreactor having improved gas yield is specified, in which the necessary residence time of the biomass in the rotting vessel is decreased. On fermentation of dry, that is to say non-pumpable, biomass, owing to the moisture present in the biomass, percolating juices, what is termed percolate, are formed and are taken off via a drainage system and, if appropriate, is recirculated from the top onto the biomass to be fermented. It has, now turned out that the biogas yield is significantly increased, in the region between 10% and 40%, when the resultant percolate is not taken off immediately via the drainage system, but is backed up in the rotting vessel up to a certain level. This is achieved in terms of the device in such a manner that the rotting vessel is designed so as to be liquid-tight, that is to say even the flap for charging and unloading the rotting vessel has to be made in a liquid-tight manner, and also must be constructed in a correspondingly stable manner in order to withstand the resultant liquid pressure. By means of the combination of the existing percolate drainage system with a percolate control unit it is possible to set the liquid level of the percolate in the biomass to be fermented and to control it in such a manner that the biogas production rate or the biogas yield is maximal.

Limitation: The patent claims that the percolate from the first digester is stored in a rotting vessel which if desirable is recirculated from the top onto the biomass. No attempt has been made to convert the rotting vessel into another methane generating digester, as also the present entire process is anaerobic.

2) U.S. Pat. No. 7,144,507 discloses “DRY CYCLE ANAEROBIC DIGESTER”.

Abstract: The present invention provides a digester for handling waste or contaminated materials. A process and an apparatus for processing are disclosed. A Dry Cycle Anaerobic Digester (DCAD) uses tanks to perform aerobic and anaerobic digestion to eliminate the waste, while producing little or no sludge.

Limitation: The invention claims handling waste in liquid form storing it for a defined period and thereby emptying the tanks followed by drying of the tank. This does not have any effect on digester size. As also the process is aerobic and anaerobic, whereas the present invention describes a dual state anaerobic process.

3) Patent Application No WO/2007/075762 discloses “ANAEROBIC PHASED SOLIDS DIGESTER FOR BIOGAS PRODUCTION FROM ORGANIC SOLID WASTES.”

Abstract: The present invention provides methods for the generation of methane by a two phase anaerobic phase system (APS) digestion of organic substrates. Also provided is a device for practicing the methods of the invention. The APS-digester system is a space-efficient, high-rate solids digestion system. The APS-digester system consists of one or more hydrolysis reactors, a buffer tank and one biogasification reactor.

Limitations: This invention describes biogas production in two stages: hydrolysis and methanization. In the first hydrolytic reactor, volatile fatty acids are produced, which are converted into biogas in the second hydrolytic reactor. Thus the process needs two reactors wherein only the second reactor is for anaerobic biomethanation.

4) Patent Application No WO 2006/017991 discloses “Stepped Sequential Treatment method for municipal domestic refuse.”

Abstract: The present invention provides a treatment method of municipal domestic refuse. In the method the organic matter processes an anaerobic fermentation; the obtained methane can be helpful to burning to generate electricity. The biogas residue from the anaerobic fermentation can be used as a culture material for growing edible mushrooms. The residue discharged from edible mushrooms can be used to cultivate earthworm. Besides the organics, the other substance of the municipal domestic refuse will be incinerated to generate electricity. The present invention realizes a comprehensive utilization of waste resource.

Limitations: The process is restricted only for municipal refuse. No treatment is specified for any other type of waste.

### BRIEF DESCRIPTION OF DRAWINGS

The present invention will be more fully understood and appreciated by reading the following detailed description in conjunction with the accompanying drawings.

FIG. 1 is the process flow diagram of dual phase digestion process using the solid state digester and the liquid state digester of the present invention in which;

### PART LIST

1) Feed Storage Tank
2) Solid State Methane Digester
3) Reaction chamber of solid state methane digester
4) A vertical perforated unit (tube)
5) Liquid State Methane Digester
6) Reaction chamber of liquid state methane digester
7) Biogas Storage Vessel (Dome)
8) Inlet port for collecting percolate
9) Outlet port for recycling the percolate
10) Culture Preparation Tank
11) Filtration Unit
12) Manure Preparation Unit
13) Spray recirculation system
14) Solid handling pump
15) Control valves/Regulators
16) Insulated Feed Inlet port
17) Leachate outlet port connected to the liquid digester
18) Digested material outlet port
19) Gas outlet port

### DISCLOSURE OF THE INVENTION

It is an object of the present invention to provide a method of biomethanation from biomass by combined solid and liquid state anaerobic fermentation.

An object of the present invention is to provide a method of biomethanation from biomass in two phases i.e. solid and liquid.

A further object of the present invention is to overcome problems associated with floating layers of scum formation which reduces the output of the biomethanation process.

A further object of the present invention is to reduce the digester size as compared to existing anaerobic digesters.

A further object of the present invention is to reduce the overall hydraulic retention time of the biomethanation process.

Still further object of the present invention is to provide a method for biomethanation of organic solid waste feeds, which utilizes minimum natural resources like water; electricity etc & can handle heterogeneous waste in the same digester scheme.

Another object of the present invention is to utilize the percolate produced through percolating units from the dry state digester biomass for further biogas production in other i.e. liquid state methane digester.

Yet another object of the present invention is to generate a self sustaining system, generation of biogas, generation of fuel, producing manure and producing minimum liquid effluent.

Yet another object of the present invention is to reduce waste water as compared to liquid state digestion systems and therefore reduce the requirement of equipment for managing the effluent stream.

Another object of the present invention is to provide a fast, economic and efficient biomethanation process.

Still another object of the present invention is to reduce the capital cost of biogas generation process.

Further object of the present invention is to reduce the processing and drying needs to use the non-digested biomass as manure or base material for organic fertilizer.

### SUMMARY OF THE INVENTION

The present invention envisages a combined dual biomethanation process and in two or more different digesters. The number of digesters depends upon the retention time of the biomass used for the biomethanation process and the choice of the designer of the system. In solid state methane digester, the one phase, bacterial cultures developed for a specific feed material and the biomass is mixed in a desired ratio by mechanical means. The leachates generated in the solid state methane digester are collected at the base of the reaction chamber of the solid state methane digester by means of percolating pipes or other appropriate mechanism. The percolates produced are re-circulated by means of sprinkler or other appropriate arrangement incorporated in the solid state methane digester and in fluid communication with the liquid state methane digester. The contents of liquid state methane digester is heated and stirred occasionally as required, therein for producing methane rich gas, wherein, both the methanogenic digesters are maintained under anaerobic conditions. The process thus maintains the required temperature for microbial activity for biogas generation in both solid state and the liquid state methane digesters due to recirculation of lechates produced in the biomethanation process. The liquid state methane digester is fed with specific cultures to convert readily degradable organic matter like sugars and volatile fatty acids into biogas. Simultaneously, heating of liquid state methane digester and recirculation of the culture into the solid state methane digester helps improve the digestion rate. Part of the sludge produced from the solid state methane digester and some portion of the slurry produced from the liquid state methane digester is carried into a culture preparation unit for use as culture for next cycle of biogas production. The remaining solids and liquid is than filtered through appropriate filtration and/or drying units for converting it into manure of desired consistency. The biogas produced from both the digesters is collected in a common gas storage unit.

The present invention results into overall reduction in retention time for biomethanation. Both the digesters are heated between temperatures of about 30 Degree Centigrade to about 40 Degree Centigrade for mesophilic cultures. The temperature range is varied based on the type of bacteria used, viz. mesophilic, thermophilic, and the like. The methane rich gas generated is collected in a gas collecting assembly. The resultant produce i.e. methane and carbon dioxide (CO₂) containing biogas may be used for cooking purposes or for generating electricity or as vehicle fuel, etc., either as is or after cleaning and/or compressing to higher pressures. This mixture can also be converted to purified methane and compressed to replace CNG and used in vehicles or other applications. It could even be introduced in natural gas pipelines to add to their existing capacity.

The said process is thus a self sustaining system generating biogas from substrate, generation of fuel, producing manure, resulting in minimum effluent slurry.

### DETAILED DESCRIPTION OF THE PREFERRED EMBODIMENTS

The foregoing objects of the said invention are accomplished and the problems and shortcomings associated with the prior art techniques are overcome by the present invention as described below in the preferred embodiment.

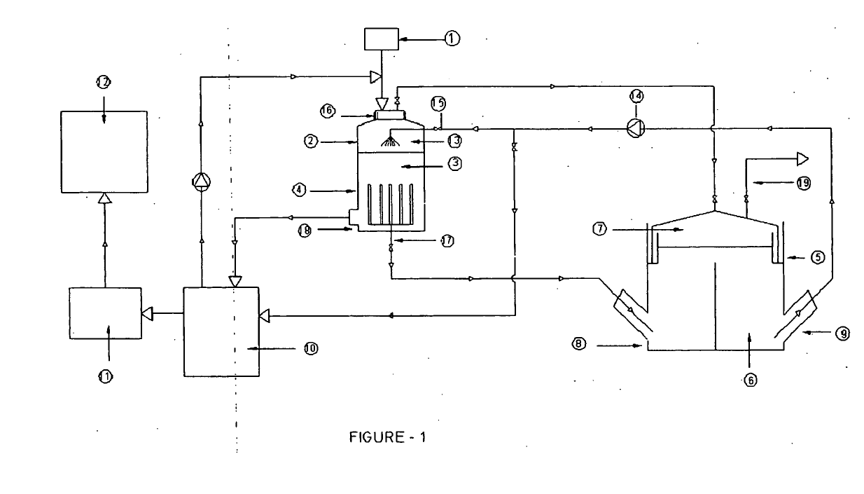

Embodiments of the invention are discussed below with reference to FIG. 1. In a preferred embodiment, the process comprises two or more anaerobic reactors or digesters a culture preparation tank, a filtration unit, a manure preparation unit and a gas storage unit. The two stages/phases i.e. solid state methanogenesis and liquid state methanogenesis are carried out separately in the said two types of digesters. The number of digesters varies depending on the retention time of the biomass used. The first and the second or the one and the other phase are carried out in presence of microorganisms. The reactors are provided with facilities for temperature control and stirring mechanism as required.

In a preferred embodiment, the process of biomethanation comprises two or more methane digesters (2), (5), gas collecting unit (7), culture preparation tank (10), filtration unit (11) and manure preparation unit (12). The solid state methane digester (2) comprising a reaction chamber (3) for conversion of biomass into biogas, a vertical perforated unit probably tube (4), spray recirculation system (13), leachate outlet port (17) is in fluid communication with the liquid state methane digester (5), digested material outlet port (18) which is in communication with the culture preparation tank (10) and a fixed gas collecting chamber. The dry feed/biomass and substrate specific culture is introduced into the solid state methane digesting tank (2) from the feed storage tank (1). The feed/biomass preferably used are agro residues, oilcakes like paddy straw; wheat straw, maize, Napier grass, press mud, castor, sal, food waste, biodegradable municipal waste and alike. The total solids of the biomass preferably are in the range of 15-20 percent. The organic solid waste is digested by addition of specially developed microbial population capable of producing required enzymes. The microbial consortium is specifically prepared for a particular feed as a target and is enriched with natural microbial mixtures such as cow-dung, sewage and the like, by a process of restricting its nutrition to the subject feed over a period of time. Once enriched, this consortium can be propagated and made available for deploying in the reactor. The said solid state methane digester (2) has a percolation unit/tube (4) internally connected in parallel to the base of the reaction chamber of said solid state methane digester (3) to facilitate percolation of lechates from the feed present in the said digester (2). The process maintains the required temperature for microbial activity for biogas generation due to recirculation of lechates produced by the biomethanation process. The lechates are heated to a desired temperature for optimum performance of the bacterial consortium present in the digesters. The organic solid waste is digested to produce biogas, which generates percolate and solid digestate. The solid digestate is further utilized for feed and manure preparation. A sprinkler and/or spray recirculation system (13) is introduced into the said solid state digester (2) which sprinkles digested slurry on top of the reaction mixture from the said liquid state methane digester (5) and collects percolate and/or lechates in a manifold created at the base of the solid state methane digester (2). The lechates produced from individual percolation unit are collected through a manifold into the said liquid state methane digester (5). The sprinkler (13) is in fluid communication with the said liquid state methane digester (5) and is suspended internally in the head space of the said solid state methane digester (2). The solid state methane digester (2) has a conduit/outlet arranged (18) at the lower region of the digesting tank for discharging sludge from the digesting tank, which is in communication with the culture preparation tank (10). The biomass is passed through the reaction chamber (3) for a period of time (about 5 to 20 days) sufficient for the feed mixture to be anaerobically digested. The period for degradation of biomass to biogas is variable, which depends on the retention time of the substrate used.

The liquid state methane digester (5) consists of a reaction chamber (6) and a flexible biogas collecting vessel preferably a dome shaped (7) for extraction of biogas generated from solid state methane digester (2) and liquid state methane digester (5). The biogas collecting vessel (7)/dome is mounted in the head region of the liquid state methane digester (5), which is movable and displaces gas by vertically upward movement. The liquid state methane digester (5) has provisions for collecting the percolate at the lower region of the digester (8) and an outlet at another lower end (9) for recycling the digested slurry into the solid state methane digester (2) by means of spray pumps and dispensers. The sludge produced is further filtered through a filtration unit (11) which may be sand filter or any other appropriate filter unit. The filtered sludge is finally dispatched for manure preparation into the manure preparation unit (12) manually. The number of solid state methane digesters varies with respect to the retention time of the biomass/substrate used for biogas generation. The spray recirculation system (13) maintains the desired temperature conditions inside the solid state methane digester (2) which in turn is controlled by solid handling pump (14). In the said liquid, state methane digester (5), the conversion into biogas is brought by housing enriched microbial consortia, leachates and/or percolates from said solid state methane digester (2). The conversion into biogas is brought by addition of enriched microbial consortia, followed by heating it between temperature ranges of about 30 Degree Centigrade to about 40 Degree Centigrade or as may be required by the consortia of microbes, with or without occasional stirring. The reactor size is optimized taking into consideration the microbial population and retention time required for digestion. Introduction of the liquid state methane digester optimizes biogas generation from the non digested slurry which otherwise is disposed off.

Valves and regulators (15) or any other appropriate flow control mechanism are introduced to control the flow of lechates/slurry. The culture preparation tank (10) is than regularly fed with the substrate and portion of the solid digested material from the solid state methane digester (2). The gas generated from the digesters is collected in storage vessel. The mixture produced in the said culture preparation tank (10) is utilized as feed further to obtain biogas.

The said non digested material prepared in the said culture preparation unit (10) is introduced partly in the said solid state methane reactor along with fresh biomass and partly treated for manure preparation. The said non digested material is introduced onto filtration tank (10) for draining excess water/liquid present in the solid digested material. The said, non digested material further is subjected for composting to manure (12) in order to achieve the desired quality of carbon to nitrogen ratio. Biogas generated from both the digesters (2, 5) is collected in a gas collecting assembly which further can be utilized for cooking purposes or generating electricity or other productive uses like vehicle fuel with or without cleaning. Biogas can also be converted to purified methane and compressed to replace CNG in vehicle. The said process reduces the hydraulic retention time for the biomethanation process to 5 to 20 days depending on the substrate. The said process also reduces water consumption by about 50 percent as to that required by conventional method of biomethanation. The said process introduces dual biomethanation process by introduction of liquid state methane digester (5).

While considerable emphasis has been placed herein on the specific steps of the preferred process and components of the preferred embodiment, and many details have been set forth for purpose of illustration, it will be appreciated that many alterations can be made and that many modifications can be made in the preferred embodiment without departing from the principles of the invention. These and other changes in the preferred embodiment as well as other embodiments of the inventions will be apparent to those skilled in the art from the disclosure herein, whereby it is to be distinctly understood that the foregoing descriptive matter is to be interpreted merely as illustrative of the invention and not as a limitation.

The invention is further described with the help of following non limiting illustrations.

### Illustration 1:

1) Fresh Napier Grass is Used as Substrate.

Fifteen kilograms of fresh Napier grass with total solids (TS) ranging from 20 to 25 percent, is pulverized upto 3 to 4 mm and allowed to undergo the said process of biomethanation.

The results observed are as follows:

| | | | Biogas | | | |
|---|---|---|---|---|---|---|
| | | | Potential | Reten- | Expected | Actual |
| | | Digester | recovery | tion | Biogas | Biogas |
| Sr. | | to Gas | (Liters/ | Time | production | production |
| No. | Substrate | Ratio | kg T.S.) | (Days) | (liters/day) | (liters/day) |
| 1 | Fresh | 1.2-1.5 | 250-350 | 18 | 900 | 1300-1400 |
| | Napier | | | | | |
| | grass | | | | | |

### Observations:

As the actual biogas output is about 50 percent more than the expected, the biogas to digester volume ratio is improved from 0.85 to about 1.2-1.5. The retention time is reduced to 18 days, whereby the conventional wet biogas digester producing biogas at similar gas potential recovery required over 25 days retention time. Thereby confirming the said invention.

### Illustration 2:

2) Dry Paddy Straw is Used as Substrate:

2.5 Kilograms of Paddy straw with total solids (TS) of about 88 to 90 percent is allowed to undergo the said biomethanation process. The following results are observed:

| | | | Biogas | | | |
|---|---|---|---|---|---|---|
| | | | Potential | Reten- | Expected | Actual |
| | | Digester | recovery | tion | Biogas | Biogas |
| Sr. | | to Gas | (Liters/ | Time | production | production |
| No. | Substrate | Ratio | kg T.S.) | (Days) | (liters/day) | (liters/day) |
| 1 | Paddy | 1.1-1.2 | 260-300 | 18 | 700 | 600-700 |
| | Straw | | | | | |
| | (dry) | | | | | |

### Observations:

The conventional wet type biogas digester producing biogas at similar gas potential recovery required over 25 days retention time, the route followed by the said process takes 18 days retention time to produce biogas. Thus confirming the said process.

### ADVANTAGES OF THE PRESENT INVENTION

The process reduces the overall hydraulic retention time required for the biomethanation process, thereby reducing the size of the biogas digester of equivalent capacity.

The process describes a solid state anaerobic fermentation, thus the invention overcomes the problem of scum formation, thereby increasing the efficiency of the biomethanation process.

The process allows use of mixed and/or multiple solid feeds as substrates for anaerobic digestion to produce biogas and manures.

The process describes dual biomethanation, wherein leachate from the solid state methane digester is utilized in liquid state methane digester to maximize biogas production.

The process produces less waste water than wet digestion systems and therefore requires less equipment for managing this effluent stream.

The process involves minimum auxiliary power/energy consumption:

1. A combined dual anaerobic process of biomethanation comprising;
two or more different anaerobic digesters for biogas generation in dual phase using mixed and/or multiple solid feeds as substrates;
a culture preparation tank/unit for preparing feed from non digested feed material for the said process;
a filtration unit/percolation unit for draining excess water from the non digested feed material; and
a manure preparation unit for preparing manure from the non digested feed material.

2. A process as claimed in claim 1, wherein the first or solid state methane digester comprises:
a gas collecting fixed dome or other device to gather biogas generated from the reaction mixture;
a reaction chamber for biomethanation process;
a vertical perforated unit (tube) at the internal base of solid state methane digester to facilitate;
a spray recirculation unit or other arrangement for circulation connected to the other or liquid state methane digester to facilitate percolation of lechates from the feed into the digester and maintaining the required temperature for the microbial activity for biogas generation;
control valves for controlling flow of biogas from the digesters into a common biogas collecting unit; and
a solid/slurry handling pump to control the flow of lechates from the liquid state methane digester to the solid state methane digester;
wherein the number of said state methane digester varies depending on the retention time of the biomass used for biomethanation process or other design constraints.

3. A process as claimed in claim 1, wherein the lechates produced from the solid state methane digester are utilized to generate biogas into another or liquid state methane digester with addition of desired microbes wherein the leachates are gathered due to the percolating structure preferably a mesh like structure present in the solid state methane digester.

4. A process as claimed in claim 3, wherein the lechates produced are recirculated through the anaerobic digesters to enchance microbial degradation of substrates and increase biogas production.

5. A process as claimed in claim 1, wherein the solid non digested feed material is collected a in culture preparation unit to produce a culture for the biomethanation process.

6. A process as claimed in claim 1, wherein manure is produced in a manure preparation unit through the step of filtration of non digested material until a desired quality of carbon to nitrogen ration is obtained.

7. A process as claimed in claim 1, wherein mixed and/or multiple solid feeds can be used as substrates for anaerobic digestion to produce biogas and manures.

8. A process as claimed in claim 1, wherein the biomethanation process reduces the overall hydraulic retention time to 5 to 20 days required for the biomethanation process, thereby reducing the size of the biogas digester by at least 50 percent of equivalent capacity.

9. A process as claimed in claim 1, wherein the substrates used include at least any of agro residues, and oilcakes.

10. A process as claimed in claim 1, wherein the first or solid state methane digester process is self sustaining producing, biogas, fuel and manure with minimum effluent slurry.

11. A process as claimed in claim 1 wherein the oilcakes include at least one of any of the following:
paddy stray, wheat straw, maize, Napier grass, press mud, castor, sal, food waste, biodegradable municipal waste and alike.
